SEQUENCE LISTING (3 sequences)

SEQ ID NO: 1 - DNA, 20 bp, Artificial Sequence
  Forward primer for detecting a SNP marker
gcttcttaccatgcacccag

SEQ ID NO: 2 - DNA, 20 bp, Artificial Sequence
  Reverse primer for detecting a SNP marker
gggcagaaagggatcacgaa

SEQ ID NO: 3 - DNA, 450 bp, Artificial Sequence
  DNA sequence of the KRT74 gene locus
gcttcttaccatgcacccagcagaggagctggtgctgcctttggaagccgaagcctctacagtctctgtagaggggatctctgtattcccctcaaggtggccggcagcagtgttcggactggaggttacaacttcaggcttagctctgggtatggagggggccggcccagcggctttgctggcagtatgtttggcagtgtggtcctggggtctgtgtgcccatccatgtgcccatccatgtgcccgcctgggggcatccaccaggtcgtcgtccacaagagcctcctggctcccctcaacgtggagctggacccggagatccagaaggtgcgcgcccaggagcgggagcagatcatggctctgaacaacaagttcgcctccttcatcgacaaggtgggtctccaggtggataagacagtccagtgagatgttcgtgatccctttctgcct

(19) United States
(12) Patent Application Publication
(10) Pub. No.: US 2023/0212694 A1
(43) Pub. Date: Jul. 6, 2023

(54) SNP MARKER RELATED TO WOOL TRAITS OF FINE-WOOL SHEEP, AND DETECTION PRIMER SET, KIT, DETECTION METHOD AND USE THEREOF

(71) Applicant: Institute of Animal Sciences of Chinese Academy of Agricultural Sciences, Beijing (CN)

(72) Inventors: Yuehui Ma, Beijing (CN); Lin Jiang, Beijing (CN); Benmeng Liang, Beijing (CN); Yabin Pu, Beijing (CN)

(73) Assignee: Institute of Animal Sciences of Chinese Academy of Agricultural Sciences, Beijing (CN)

(21) Appl. No.: 17/605,734

(22) PCT Filed: Jun. 28, 2021
(86) PCT No.: PCT/CN2021/102648

§ 371 Date: Oct. 22, 2021

(30) Foreign Application Priority Data
Sep. 22, 2020 (CN) 202011000826.X

(51) Int. Cl. C12Q 1/6888

(52) U.S. Cl. CPC C12Q 1/6888; C12Q 2600/124; C12Q 2600/156

(57) ABSTRACT

Disclosed is an SNP marker related to wool traits of a fine-wool sheep, and a detection primer set, a detection method and use thereof, which belong to the technical field of molecular marker detection; the SNP marker is located at 133486008bp on sheep chromosome 3. There is an A/C base mutation at the site, which is significantly related to the wool traits of fine-wool sheep. The SNP marker of the present disclosure may be used in molecular marker assisted breeding of sheep. The molecular marker provided by the present disclosure is not limited by the age, sex, etc. of the sheep, may be used for fine-wool sheep breeding (even a sheep at birth may be accurately screened), and significantly promotes the breeding process of fine-wool sheep.

### CROSS REFERENCE TO RELATED APPLICATION

This patent application claims the benefit and priority of Chinese Patent Application No. 202011000826.X filed on Sep. 22, 2020, the disclosure of which is incorporated by reference herein in its entirety as part of the present application.

### TECHNICAL FIELD

The present disclosure relates to the technical field of molecular marker detection, in particular to an SNP marker related to wool traits of fine-wool sheep, and a detection primer set, a kit, a detection method and use thereof.

### BACKGROUND ART

KRT74 (Keratin, type II cytoskeletal 74) gene is one of the key genes regulating the development of hair follicles. It exists in skin tissue and is specifically expressed in the Huxley layer of the inner root sheath (IRS) of the hair follicle. It plays an important role in maintaining the structure of the hair follicle. The mutation of this gene affects the formation of keratin intermediate filaments, causing autosomal dominant wool hair (ADWH) syndrome, which is characterized by tightly curly hair. Selection signal screening in fine-wool sheep, semi-fine-wool sheep and Mongolian sheep populations indicated that the polymorphic variation of the KRT74 gene locus had a significant effect on the wool traits of fine-wool sheep.

Fine-wool sheep is famous for its natural fiber wool, which is used in the textile industry and is an important agricultural product. The quality and yield of wool depend on the development of hair follicles. The development of hair follicles is a complex biological process involving many different proteins. Early research found that several important genes were related to wool traits, including fiber diameter, wool color, crimp frequency (wave number per centimeter of wool fiber length), and wave characteristics. However, due to the low level of selection and breeding of fine-wool sheep, the lack of breeding markers, and the low degree of development and utilization, the resource advantage of fine-wool sheep cannot be transformed into economic advantage. Therefore, it is necessary to develop specific molecular markers for fine-wool sheep and apply them to the breeding of fine-wool sheep.

### SUMMARY

The objective of the present disclosure is to provide an SNP marker related to wool traits of a fine-wool sheep, and a detection primer set, a kit, a detection method and use thereof.

In order to achieve the objective, a technical scheme is provided in the present disclosure as follows:

The present disclosure provides an SNP marker related to wool traits of a fine-wool sheep. The SNP molecular marker is located at site 133486008bp on sheep chromosome 3 ; there is an A/C base mutation at the site, which is significantly related to the wool traits of fine-wool sheep; the SNP molecular marker is based on a sheep genome with version number is Oar_v4.0, November 2015.

The present disclosure further provides a primer set for detecting the SNP marker in the above scheme, including a forward primer and a reverse primer; the nucleotide sequence of the forward primer is shown as SEQ ID No. 1; the nucleotide sequence of the reverse primer is shown as SEQ ID No. 2.

The present disclosure further provides a kit for detecting the SNP marker in the above scheme, which comprises the primer set forth in the above scheme.

In some embodiments, the kit may further include dNTPs, a Phanta Max Super-Fidelity DNA polymerase and a PCR reaction buffer.

In some embodiments, the kit may further include a standard positive template.

The present disclosure further provides a method for detecting the SNP marker in the above scheme, which includes the following steps:
1) extracting a genomic DNA of a sheep to be tested;
2) using the genomic DNA of the sheep to be tested as a template, performing a PCR amplification with the primer set forth in the above scheme to obtain a PCR amplification product; and
3) detecting a genotype at 274 bp of the PCR amplified product to obtain a genotype of the SNP site.

In some embodiments, a 25 µl system of the PCR amplification in step 2) may include the following components: 1 µl of genomic DNA of the sheep to be tested, 1 µl of forward primer and reverse primer, 0.5 µl of dNTP mix, 0.5 µl of Phanta Max Super-Fidelity DNA polymerase, 12.5 µl of 2×PCR reaction buffer and a balance of double distilled water;

A concentration of the genomic DNA of the sheep to be tested is 50-100 ng/µl; concentration of the forward primer and the reverse primer are respectively 10 pmol/µl; and a concentration of the dNTP mix is 10 mmol/L.

In some embodiments, procedures of the PCR amplification in step 2) may include: a pre-denaturation at 95° C. for 5 minutes; a total of 35 cycles of denaturation at 95° C. for 30 seconds, annealing at 60° C. for 30 seconds, extension at 72° C. for 2 minutes; and an incubation at 72° C. for 2 minutes.

The present disclosure further provides a use of the SNP marker or the primer set or the kit or the method in the above scheme in sheep molecular marker assisted breeding.

The beneficial effect of the embodiments may include: disclosed is an SNP marker related to wool traits of a fine-wool sheep, which is located at 133486008bp on sheep chromosome 3 ; there is an A/C base mutation at the site, which is significantly related to the wool traits of fine-wool sheep. The SNP marker of the present disclosure may be used in molecular marker assisted breeding of sheep. The molecular marker provided by the present disclosure is not limited by the age, sex, etc. of the sheep, may be used for fine-wool sheep breeding (even a sheep at birth may be accurately screened), and significantly promote the breeding process of fine-wool sheep.

### BRIEFT DESCRIPTION OF THE DRAWINGS

FIG. 1 is a diagram showing sequencing peaks of the three genotypes; wherein (a) is AA type; (b) is CC type; (c) is AC type; and

FIG. 2 shows a validation result of the SNP site in an enlarged population.

### DETAILED DESCRIPTION OF THE EMBODIMENTS

The present disclosure provides an SNP marker related to wool traits of a fine-wool sheep. The SNP molecular marker is located at site 133486008bp on sheep chromosome 3. There is an A/C base mutation at the site, which is significantly related to the wool traits of fine-wool sheep; there are three genotypes (CC, CA and AA) at the locus and the proportion of wool traits of individual fine-wool sheep with CC genotype is significantly higher than that of CA genotype and AA genotype; the SNP molecular marker is based on a sheep genome with version number is Oar_v4.0, November 2015.

In the present disclosure, at site 133486008bp on sheep chromosome 3, frequencies of allele C in fine-wool sheep and semi-fine-wool sheep are as high as 93.1% and 98.75% respectively, while in coarse-wool sheep, the frequency is only 34.2%. It has great guiding significance for distinguishing and screening of wool traits of fine-wool sheep by genotype, and may improve the accuracy and efficiency of sheep wool screening.

The present disclosure further provides a primer set for detecting the SNP marker in the above scheme, including a forward primer and a reverse primer; the nucleotide sequence of the forward primer is shown as SEQ ID No. 1, specifically: 5′-gcttcttaccatgcacccag-3′; the nucleotide sequence of the reverse primer is shown as SEQ ID No. 2, specifically: 5′- aggcagaaagggatcacgaa-3′.

The present disclosure further provides a kit for detecting the SNP marker in the above scheme, which comprises the primer set in the above scheme.

In the present disclosure, the kit further includes dNTPs, a Phanta Max Super-Fidelity DNA polymerase and a PCR reaction buffer.

In the present disclosure, the kit may further include a standard positive template, the genotype of the standard positive template is CC at 133486008bp; the DNA of the standard positive template is used as a positive reference to increase the detection accuracy of the SNP site.

The present disclosure further provides a method for detecting the SNP marker in the above scheme, which includes the following steps:
1) extracting a genomic DNA of a sheep to be tested;
2) using the genomic DNA of the sheep to be tested as a template, performing a PCR amplification with the primer set forth in the above scheme to obtain a PCR amplification product; and
3) detecting a genotype at the 274 bp of the PCR amplified product to obtain the genotype of the SNP site.

In the present disclosure, a genomic DNA of a sheep to be tested is firstly extracted; there are no special limitations on the method for extracting a genomic DNA of a sheep, a conventional method in the art may be adopted.

After the genomic DNA of the sheep to be tested is obtained, the genomic DNA of the sheep to be tested is used as a template and a PCR amplification with the primer set forth in the above scheme is performed to obtain a PCR amplification product.

In the present disclosure, a 25 µl system of the PCR amplification in step 2) may include the following components: 1 µl of genomic DNA of the sheep to be tested, 1 µl of forward primer and reverse primer, 0.5 µl of dNTP mix, 0.5 µl of Phanta Max Super-Fidelity DNA polymerase, 12.5 µl of 2×PCR reaction buffer and a balance of double distilled water. In the present disclosure, a concentration of the genomic DNA of the sheep to be tested may preferably be 50-100 ng/µl, and more preferably 60-80 ng/µl; a concentration of the forward primer and reverse primer may preferably be 10 pmol/µl; and the concentration is preferably 10 mmol/L.

In the present disclosure, procedures of the PCR amplification may preferably include: a pre-denaturation at 95° C. for 5 minutes; a total of 35 cycles of denaturation at 95° C. for 30 seconds, annealing at 60° C. for 30 seconds, extension at 72° C. for 2 minutes; and an incubation at 72° C. for 2 minutes.

After the PCR amplification product is obtained, a genotype at 274 bp of the PCR amplified product is detected to obtain a genotype of the SNP site.

The method for detecting the genotype of PCR amplified products is not particularly limited in the present disclosure, and a conventional genotype detecting method in the art can be adopted. During the specific implementation of the present disclosure, the Sanger sequencing method may be used to detect the genotype of the sheep to be tested

The method of the present disclosure may have the advantages of accuracy, reliability and simple operation.

The present disclosure further provides a use of the SNP marker or the primer set or the kit or the method in the above scheme in sheep molecular marker assisted breeding. The SNP marker or the primer set or the kit of the present disclosure may be combined with other specific primers or kits for detecting the phenotype of Chinese sheep for Chinese sheep classification and breeding research.

The technical schemes of the present disclosure will be clearly and completely described below in conjunction with the examples of the present disclosure. Obviously, the described examples are only a part of the examples of the present disclosure, rather than all the examples. Based on the examples of the present disclosure, all other examples obtained by those of ordinary skill in the art without creative work shall fall within the protection scope of the present disclosure.

### Example 1: Identification of Polymorphism of Sheep KRT74 Gene

### 1.1 Extraction of the Genomic DNA of Chinese Sheep to Be Tested

The genomic DNA of tissue samples from 84 semi-fine-wool sheep (20 from Liangshan, 24 from Guizhou and 40 from Yunnan, China), 165 fine-wool sheep (84 from Xinjiang, 20 from Qinghai, 21 from Gansu, 20 from Inner Mongolia, and 20 from Jilin, China) and 39 Mongolian sheep from Qinghai (China) was extracted by the magnetic bead method.

### 1.2 Amplification of Nucleotide Sequences Containing the SNP Site

Primers were designed based on the sequence of the KRT74 gene locus included in the NCBI database, including a forward primer F: 5′-gcttcttaccatgcacccag-3′, as shown in SEQ ID NO. 1; and a reverse primer R: 5′-aggcagaaagggatcacgaa-3′, as shown in SEQ ID NO.2. The nucleotide sequence where the SNP is located, as shown in SEQ ID NO.3, in particular:

gcttcttaccatgcacccagcagaggagctggtgctgcctttggaagccg
aagcctctacagtctctgtagaggggatctctgtattcccctcaaggtgg
ccggcagcagtgttcggactggaggttacaacttcaggcttagctctggg
tatggagggggccggcccagcggctttgctggcagtatgtttggcagtgt
ggtcctggggtctgtgtgcccatccatgtgcccatccatgtgcccgcctg
ggggcatccaccaggtcgtcgtccacaagagcctcctggctcccctcaac
gtggagctggacccggagatccagaaggtgcgcgcccaggagcgggagca
gatcatggctctgaacaacaagttcgcctccttcatcgacaaggtgggtc
tccaggtggataag acagtccagt gagatgttcg tgatcccttt
ctgcct,

was amplified. The SNP site was located at 274 bp of the PCR amplified fragment (shown in SEQ ID NO. 3), where the base is A or C.

Wherein, a 25 µl system of the PCR amplification included 100 ng/µl template DNA 1 µl, 10 pmol/µl forward primer and reverse primer 1 µl each, 10 mmol/L dNTP mix 0.5 µl, Phanta Max Super-Fidelity DNA polymerase 0.5 µl, 2×PCR reaction buffer 12.5 µl, and a balance of double distilled water.

Wherein, the procedures of the PCR amplification included a pre-denaturation at 95° C. for 5 minutes; a total of 35 cycles of denaturation at 95° C. for 30 seconds, annealing at 60° C. for 30 seconds, extension at 72° C. for 2 minutes; and an incubation at 72° C. for 2 minutes.

### 1.3 Detection of PCR Amplified Fragments to Obtain the SNP Marker

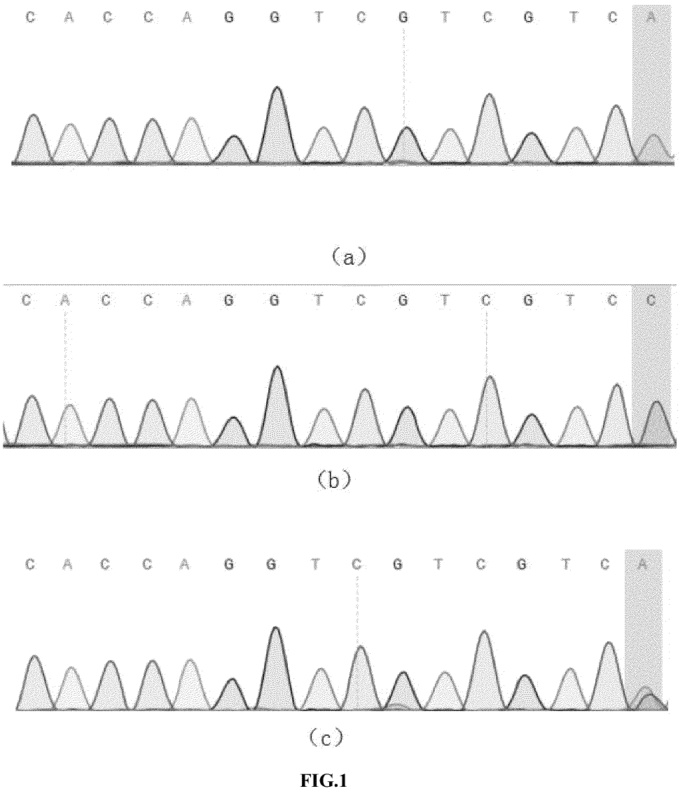

The PCR amplification product in 1.2 was sequenced and verified, and the genotype at 274 bp of the PCR amplification product was the genotype of the SNP site. The sequencing peak diagram of the three genotypes was shown in FIG. 1.

### Example 2

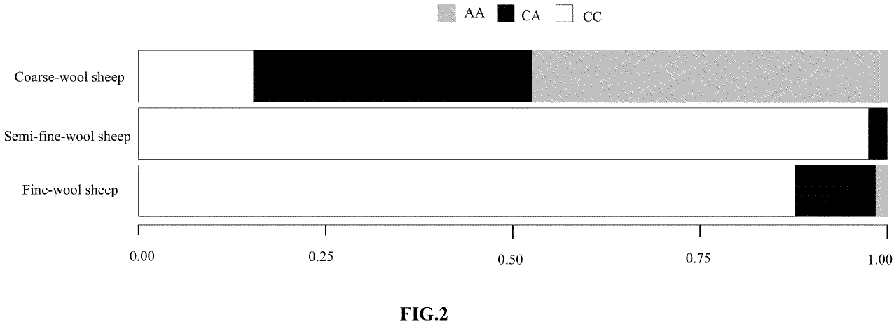

668 Chinese sheep was used to perform the analysis of polymorphism of the KRT74 locus in enlarged population. According to the test and analysis by T test, the frequency of the allele C in the fine-wool sheep and semi-fine-wool sheep is significantly higher than that in the coarse-wool sheep (P <7.64e-12). The correlation between the allele C of the SNP site and the wool traits of fine-wool sheep was further verified (as shown in Table 1 and FIG. 2). It could be seen from Table 1 and FIG. 2 that “CC” was the dominant genotype of the fine-wool sheep breed, while “AA” and “CA” were the genotypes in the coarse-wool sheep breed.

TABLE 1

Genotype frequency and allele frequency of SNP locus in fine-wool, semi-fine-wool and coarse-wool sheep

| Phenotype | CC | CA | AA |
|---|---|---|---|
| Fine-wool sheep | 179 | 22 | 3 |
| Semi-fine-wool sheep | 78 | 2 | — |
| coarse-wool sheep | 59 | 143 | 182 |

The above are only the preferred embodiments of the present disclosure. It should be pointed out that for those of ordinary skill in the art, without departing from the principle of the present disclosure, several improvements and modifications can be made, and these improvements and modifications shall be also regarded as within the protection scope of the present disclosure.

What is claimed is:

1-10. (canceled)

11. A primer set for detecting an SNP marker, comprising a forward primer and a reverse primer;
wherein the nucleotide sequence of the forward primer is shown as SEQ ID No. 1;
wherein the nucleotide sequence of the reverse primer is shown as SEQ ID No. 2;
wherein the SNP marker relates to wool traits of a fine-wool sheep, and the SNP molecular marker is located at site 133486008bp on sheep chromosome 3;
wherein there is an A/C base mutation at the site, which is significantly related to the wool traits of fine-wool sheep; and
wherein the SNP molecular marker is based on a sheep genome with version number is Oar_v4.0, November 2015.

12. A primer set according to claim 11, wherein the primer set comprises a kit for detecting an SNP marker.

13. The primer set according to claim 12, wherein the kit further comprises dNTPs, a Phanta Max Super-Fidelity DNA polymerase and a PCR reaction buffer.

14. The primer set according to claim 12, wherein the kit further comprises a standard positive template.

15. A method for detecting an SNP marker comprising:
a) extracting a genomic DNA of a sheep to be tested;
b) using the genomic DNA of the sheep to be tested as a template, performing a PCR amplification with a primer set to obtain a PCR amplification product; and
c) detecting a genotype at 274 bp of the PCR amplified product to obtain a genotype of the SNP site;
wherein the SNP marker relates to wool traits of a fine-wool sheep, and the SNP molecular marker is located at site 133486008bp on sheep chromosome 3;
wherein there is an A/C base mutation at the site, which is significantly related to the wool traits of fine-wool sheep;
wherein the SNP molecular marker is based on a sheep genome with version number is Oar_v4.0, November 2015;
wherein the primer set comprises a forward primer and a reverse primer;
wherein the nucleotide sequence of the forward primer is shown as SEQ ID No. 1; and
wherein the nucleotide sequence of the reverse primer is shown as SEQ ID No. 2.

16. The method according to claim 15, wherein a 25µl system of the PCR amplification in step (b) comprises:
1 µl of genomic DNA of the sheep to be tested;
1 µl of forward primer and reverse primer;
0.5 µl of dNTP mix;
0.5 µl of Phanta Max Super-Fidelity DNA polymerase;
12.5 µl of 2×PCR reaction buffer; and
a balance of double distilled water;
wherein the concentration of the genomic DNA of the sheep to be tested is 50-100 ng/µl;
wherein the concentration of the forward primer and the reverse primer are 10 pmol/µl; and
wherein the concentration of the dNTP mix is 10 mmol/L.

17. The method according to claim 15, wherein procedures of the PCR amplification in step (b) comprises:
a pre-denaturation at 95° C. for 5 minutes;
a total of 35 cycles of denaturation at 95° C. for 30 seconds;
annealing at 60° C. for 30 seconds;
extension at 72° C. for 2 minutes; and
an incubation at 72° C. for 2 minutes.

18. A method for analyzing a polymorphism of a KRT74 locus in sheep molecular marker assisted breeding, comprising using the primer set according to claim 11.

19. The method according to claim 18, wherein the sheep molecular marker assisted breeding comprises fine-wool sheep variety breeding.

20. The primer set according to claim 14, wherein the kit further comprises dNTPs, a Phanta Max Super-Fidelity DNA polymerase and a PCR reaction buffer.

21. The method according to claim 19, comprising the step of using a kit comprising dNTPs, a Phanta Max Super-Fidelity DNA polymerase and a PCR reaction buffer.

22. The method according to claim 21, wherein the kit further comprises a standard positive template.

23. The method according to claim 18, wherein the method comprises detecting the SNP marker by the steps of:
a) extracting a genomic DNA of a sheep to be tested;
b) using the genomic DNA of the sheep to be tested as a template, performing a PCR amplification with a primer set to obtain a PCR amplification product; and
c) detecting a genotype at 274 bp of the PCR amplified product to obtain a genotype of the SNP site;
wherein the primer set comprises a forward primer and a reverse primer;
wherein the nucleotide sequence of the forward primer is shown as SEQ ID No. 1; and
wherein the nucleotide sequence of the reverse primer is shown as SEQ ID No. 2.

24. The method according to claim 23, wherein procedures of the PCR amplification in step (b) comprises:
a pre-denaturation at 95° C. for 5 minutes;
a total of 35 cycles of denaturation at 95° C. for 30 seconds,
annealing at 60° C. for 30 seconds, extension at 72° C. for 2 minutes; and
an incubation at 72° C. for 2 minutes.
